(19) United States
(12) Patent Application Publication
(10) Pub. No.: US 2006/0271098 A1
(43) Pub. Date: Nov. 30, 2006

(54) Embolic filter device and related systems and methods

(76) Inventor: James C. Peacock III, San Carlos, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/414,157

(22) Filed: Apr. 28, 2006

Related U.S. Application Data
(63) Continuation of application No. PCT/US04/36415, filed on Oct. 28, 2004.
(60) Provisional application No. 60/515,282, filed on Oct. 28, 2003.

(51) Int. Cl. A61M 29/00

(52) U.S. Cl. 606/200

(57) ABSTRACT

An embolic filter system is provided that has a bioactive surface, such as locally on the surface itself or via elution into surrounding environs, and such as to debulk its filtered contents or prevent thrombosis or thromboemboli. An engineered wall provides for enhanced porosity for improved combination of blood flow through the filter and size of particulate that may be captured. Manufacturing methods are provided for improved filter assemblies, and a tether system is provided for improved in-situ deployment. A proximal filter assembly is used to debulk contents of a distal embolic filter assembly before it is removed from the patient.

### CROSS-REFERENCE TO RELATED APPLICATIONS

This application claims priority from, and is a 35 U.S.C. § 111(a) continuation of, co-pending PCT international application serial number PCT/US2004/036415, filed on Oct. 28, 2004, incorporated herein by reference in its entirety, which designates the U.S., which claims priority from U.S. provisional application Ser. No. 60/515,282, filed on Oct. 28, 2003, wherein is herein incorporated in its entirety by reference.

### STATEMENT REGARDING FEDERALLY SPONSORED RESEARCH OR DEVELOPMENT

Not Applicable

### REFERENCE TO A COMPUTER PROGRAM APPENDIX

Not Applicable

### FIELD OF THE INVENTION

The present invention is a system and method for filtering emboli from fluid flowing through a body lumen in a patient. More specifically, it is a distal embolic filter system and method providing an engineered porosity, and also providing reduction of emboli or biologic materials related to the filter.

### BACKGROUND

Embolic filters have been widely used for over a decade, principally for vena cava use in protecting against venous emboli. More recent emerging devices and applications have included arterial filters. Arterial embolic filters in particular are designed for the intended use for filtering emboli released during or contemporaneous with interventional procedures. Arterial embolic filters include both distal filters and “proximal” filter systems and methods, described in further detail below.

One particular area where distal embolic filtering has been investigated involves distal protection against emboli flowing toward the brain during carotid artery interventions, such as endarterectomy, angioplasty, stenting, or atherectomy or rotational ablation. Another area under relatively intense investigation involves filtering emboli from distal run off during or following recanalization of grafts, such as coronary bypass grafts. Peripheral vascular recanalization and stenting, such as for example of the superficial femoral artery (SFA), is also becoming a more prevalent setting where distal embolic protection is evolving with promise to become a standard of care in many circumstances.

Many distal embolic protection systems and methods provide a filter pre-disposed on a distal end portion of a guidewire chassis. The guidewire and filter are positioned translumenally through and across the intervention site, typically in an antegrade fashion, so that the filter is positioned downstream from the occlusion to be recanalized. Then the filter is deployed, generally as an expanded cage or porous material that allows blood to pass but for emboli of a predetermined size (according to the passage ports, e.g. through pores or other openings in the filter). The intervention upstream from the filter releases emboli that flow downstream into the deployed filter where they are caught. After the intervention is complete, a mechanism is provided that allows the filter to be adjusted for withdrawal, including capturing the emboli caught.

Further examples of devices and methods that provide additional background helpful in understanding the overall context of the present invention are provided in the following U.S. patents: U.S. Pat. No. 6,027,520 to Tsugita et al.; U.S. Pat. No. 6,042,598 to Tsugita et al.; U.S. Pat. No. 6,168,579 to Tsugita; U.S. Pat. No. 6,270,513 to Tsugita et al.; U.S. Pat. No. 6,277,139 to Levinson et al.; and U.S. Pat. No. 6,319,242 to Patterson et al. Additional examples are disclosed in the following Published International PCT Patent Applications: WO 00/67664 to Salviac Limited; WO 01/49215 to Advanced Cardiovascular Systems, Inc.; WO 01/80777 to Salviac Limited; and WO 02/43595 to Advanced Cardiovascular Systems, Inc. The disclosures of these references are herein incorporated in their entirety by reference thereto.

Some of the previously disclosed embolic filter approaches provide an expandable cage consisting of a braided network of crossing metal struts. As deployed in-vivo, fluid is allowed to flow through spaced gaps between the struts. Other previously disclosed embolic filters use porous membrane materials and rely on the porosity of the material itself to provide for the filtered flow therethrough. Still other previous techniques have used mechanical tools or other discrete drilling techniques to “poke” holes through membrane materials to achieve the desired pore.

A variety of encouraging clinical trials and related recent approvals for embolic filters have been published, and clinical use of various previously disclosed embolic filters is growing. However, some questions and concerns still remain, and there is great opportunity for beneficial improvement in next generations of approaches.

In one particular regard, it has been suggested in certain circumstances that thrombus material found in some filters removed after a procedure may not, in fact, represent thromboemboli caught by the filter, but rather may have been caused by the filter itself.

More specifically, one remaining concern is that the filter material spanning the vessel may actually itself provide a nidus for platelet adhesion and thrombus formation. However, a commercially viable filter has not been provided that modifies the filter material surface to specifically enhance its biocompatibility or thrombus resistance.

A concern also remains that the hemodynamics of blood flowing through the filter may be substantially compromised to the extent causing hemolysis, a widely known precursor to a thrombotic cycle. Such hemodynamic compromise may be caused, in one regard, by the size of flow pores themselves. The choice of pore size is generally determined by two considerations: (a) a maximum pore size criterium sufficient to limit passage of the minimum size of emboli to be desirably prevented from passing; and (b) a minimum pore size criterium sufficient to provide the necessary flow to perfuse the downstream circulation with minimum flow disruption and hemolysis. Another closely related consideration is the relationship between the pores and the relatively impenetrable filter material bordering those pores.

For example, in one particular regard this concerns the density of the pores (or spacing therebetween) across a unit area of filter material spanning across the flowing fluid. The relationships between the pore sizes, their shape, relative pattern and arrangement between them, and the density of the pores per unit area of wall material—any or all of these may play significant factors in the fluid dynamics and effects on blood flow and hemolysis or thrombosis in particular during filtering procedures.

Among the concerns noted above, these relate in one sense to thrombus being formed and captured, e.g. removed, by filters. In another sense, however, additional concern relates to possible thrombosis on the opposite or “back” (e.g. distal or downstream) surface of the filter. For example, in prior experience with compromised flow dynamics through resistive implants (e.g. heart valves), thrombus formation has been observed in particular on the back-side of devices. More specifically, when fluid flows across an obstruction, eddy currents form wherein fluid swirls around and behind the device. This is caused by a negative pressure or vacuum formed behind the device, such as for example according to the Bernoulli principle upon which modern aircraft wings is based due to “lift” formed by such pressure drop. In this setting, and in this location behind the obstruction, red blood cells may lyse. Notwithstanding this understanding from other fields, little has been done in the setting of filter membranes and other porous wall filters to engineer improvements against the potential thrombotic effects of lysis behind the filter membrane and surrounding the pores.

In another regard, notwithstanding whether thrombus forms on the device itself, the possible hemolysis caused by compromised fluid dynamics may still cascade to thrombotic events downstream of the filter. However, no substantial efforts have provided a filter system or method that provides protection against such downstream results.

In addition to the foregoing opportunities to improve upon previously disclosed embolic filter technologies, it also remains the case across the field that contents captured within a filter (whether or not they were formed by the filter) require removal. Accordingly, the filters when collapsed to capture their contents for removal from a patient may have substantially larger profiles following a procedure then when they were delivered to initiate the procedure. This may require a certain minimum size of sheath through which the engorged filter may be withdrawn, or may require removal of the whole transcatheter system in some circumstances if the filter will not fit through coaxial delivery catheters or cannulas. In another regard, these contents, by there very presence in the filter, provide yet further compromise to fluid dynamics through the filter while it remains indwelling in a vessel. This may provide yet a further nidus where clot may form. Notwithstanding the foregoing, short of capturing materials within the filter as a “trap” and removing them by withdrawing the filter, prevailing embolic filter technologies have not been provided with the ability to dissolve or otherwise debulk their contents prior to removal.

As mentioned above, “proximal embolic filters” is another field that has emerged in generally competitive efforts with distal embolic filters, with certain shared target markets. In the typical “proximal” approach, rather than filtering blood flow that continues to run distally from a location where an intervention is done, a complete occlusion is created distal to the intervention and stops all distal flow. Such is accomplished for example by use of a balloon located distally from a site of carotid stenting for example. Following the intervention, a system located proximally of the intervention, e.g. in the proximal carotid artery, uses suction to reverse flow in the vessel to proximally remove the contents caught within the distally occluded vessel, and aspirates those contents from the patient. Like some of the distal embolic filter experience, early data for proximal filtering appears very promising. However, also like the other prior distal filter approaches, these initial proximal filter systems and techniques also present certain shortcomings and otherwise opportunities for beneficial improvement. In one regard, for example, the filtered vessel requires complete blockage and occlusion from initiation of the procedure and until the time window for desired filtering expires. This is a ground for substantial concern in many circumstances.

Notwithstanding the respective benefits and shortcomings of the previously disclosed systems and methods for both proximal and distal embolic filtering, respectively, a prior commercial effort is not known that combines proximal filtering devices and techniques to remove emboli with distal embolic filters that capture the emboli. A need still exists for such a novel combination, which would provide the substantial combination of benefits that include: (a) filtering emboli without interrupting blood flow, plus (b) removing the filtered contents fluidically and prior to removal of the filter that may be collapsed and removed in a low profile fashion. Moreover, by providing such systems in combined form, filtering and removal may be cycled during a procedure, removing captured contents earlier while the filter beneficially remains in a cleaner, less encumbering form for an improved mode of on-going, in-dwelling use.

A need exists for a system and method that provide such a coordinated combination of proximal and distal embolic filtering features, and the improvements and benefits concomitant therewith.

A need still remains exists for distal embolic filter that is able to reduce or remove the captured contents and emboli during on-going filtering or otherwise prior to removal of the filter.

A need also still exists for an improved ability to impart to a filter wall membrane an engineered porosity that is not inherent within the membrane in order to provide improved filtering results, and in particular for filtering emboli from blood such as during vascular interventions.

A need also still exists for improved filter surfaces that enhance the filter's biocompatibility in the setting of compromised fluid dynamics, and in particular in the setting of compromised blood flow, and still more particularly in the setting where hemolysis may be prevalent in order to prevent thrombus formation on the filter surface.

A need also still exists for improved filter surfaces that elute bioactive agents that provide beneficial biological results, such as to prevent thrombus formation at or downstream of the filter, or to debulk the filter such as via thrombolytic agents or calcium dissolving agents.

### SUMMARY OF THE INVENTION

One aspect of the present invention is an embolic filter system that includes a delivery member with an elongate body, and also a distal embolic filter assembly. The filter assembly includes a wall that is adapted to be delivered to a and span across a distal location within a vessel in a patient and that is substantially porous so as to filter emboli from antegrade blood flowing to and through the wall at the distal location. The wall is mounted on a super-elastic, nickel-titanium frame that is secured to the elongated body. The frame has a memory in a radially expanded condition, and is self-expandable from a radially collapsed condition to a radially expanded condition. The frame is held in radial confinement in the radially collapsed condition by at least one releasable circumferential tether that holds the frame substantially tight around the elongated body of the delivery member. The tether is releasable at the distal location to thereby remove the radial confinement on the frame and allow the frame to self-expand to the radially expanded condition.

Another aspect of the invention is an embolic filter system that provides a distal embolic filter assembly with a wall that is adapted to be delivered to a and span across a distal location within a vessel in a patient and that is substantially porous so as to filter emboli from antegrade blood flowing to and through the wall at the distal location. This aspect includes a plurality of discrete apertures through the wall and providing the substantial porosity. Each of the plurality of apertures comprises a geometry with length being at least about twice the width, and further with the width being equal to or less than about 120 microns.

Another aspect of the invention is an embolic filter system that includes a distal embolic filter assembly with a wall that is adapted to be delivered to a and span across a distal location within a vessel in a patient and that is substantially porous so as to filter emboli from antegrade blood flowing to and through the wall at the distal location. According to this aspect, however, the filter wall comprises a composite structure with a polymer membrane in combination with a network of structural support struts. The network of structural support struts is coupled to the membrane. A plurality of apertures communicate through the membrane. At least one of the structural support struts spans across each of the apertures.

Another aspect of the invention is an embolic filter system that includes a distal embolic filter assembly with a wall that is adapted to be delivered to a distal location within a vessel in a patient and that is substantially porous so as to filter emboli from antegrade blood flowing to and through the wall at the distal location and without substantially compromising hemodynamics of the antegrade blood flow sufficient to cause substantial hemolysis. According to this aspect, however, a proximal filter assembly is also provided with an aspiration catheter and that is adapted to be fluidically coupled to the distal filter assembly at the distal location and to reverse flow at the distal location so as to aspirate contents captured on an upstream side of the embolic filter and from the patient.

Another aspect of the invention is an embolic filter system that includes a distal embolic filter assembly with a wall that is adapted to be delivered to and span across a distal location within a vessel in a patient and that is substantially porous so as to filter emboli from antegrade blood flowing to and through the wall at the distal location. According to this aspect, the wall comprises a polymeric membrane and a surface with a bioactive agent coupled to the surface and that may be different than the underlying material of the membrane. The bioactive agent is provided in a manner expressing substantial bioactivity with respect to blood in contact with the surface.

Another aspect of the invention is an embolic filter system that includes distal embolic filter assembly with a wall that is adapted to be delivered to and span across a distal location within a vessel in a patient and that is substantially porous so as to filter emboli from antegrade blood flowing to and through the wall at the distal location. According to this aspect, the wall is provided with a composite structure with a first layer on a first side comprising a membrane constructed from a first material, and also with a second layer comprising a second material deposited onto the first material. At least one of the first and second materials not inherently porous to the extent sufficient to provide the substantial porosity for embolic filtering and substantially uncompromised blood flow therethrough. A pattern of perfusion pores communicate through the first and second materials. Moreover, the first and second materials are characterized as being substantially different such that the first material if exposed within the pores to an ablation source would ablate, but whereas the same exposure is not ablative to the second material.

According to one further mode of various of these system aspects of the invention, a delivery member is provided with an elongate body. The distal embolic filter assembly is coupled to the delivery member for delivery to the distal location.

In one embodiment according to this mode, the delivery member includes a guidewire tracking member and is adapted to track over a guidewire to the distal location.

In another embodiment, the delivery member comprises an adjustable lock that is adjustable between an open condition, wherein the delivery member is adapted to track over a guidewire, to a locked condition, wherein the delivery member is adapted to lock onto the guidewire such that the guidewire and filter assembly are adapted to be removed from the patient together through a delivery sheath. According to a further embodiment, the delivery member comprises a distal delivery assembly and a detachable proximal delivery assembly coupled to the distal delivery assembly at a detachable joint. The distal embolic filter assembly is coupled to the distal delivery assembly. The distal delivery assembly is adapted to be positioned entirely within the patient, and the proximal delivery assembly is adapted to extend exernally of the patient, and the proximal delivery assembly is adapted to be released from the distal delivery assembly, when the distal embolic filter assembly is positioned at the distal location and when the adjustable lock is locked onto the guidewire. In one further highly beneficial variation, the detachable joint is of the electrolytically detachable type.

According to another mode related to the foregoing system aspects of the invention, a plurality of discrete apertures communicate through the wall and provide the substantial porosity necessary to provide for appropriate combination of substantially non-hemolytic blood flow and particulate capturing. Each of the plurality of apertures comprises a geometry with a length and a width, the length being at least about twice the width. According to still a further mode, the width is equal to or less than about 120 microns.

According to another mode, the length is equal to or greater than 120 microns.

According to another mode, the width is less than or equal to about 100 microns.

According to another mode, the width is less than or equal to about 80 microns.

According to another mode, the width is less than or equal to about 60 microns.

According to another mode, the plurality of apertures comprises at least one elongate groove through the wall and bridged by metal filaments. The geometry is defined by distance between the lateral edges of the groove and the spacing between the filaments.

In one embodiment of this mode, the system further includes a plurality of these grooves. Each extends longitudinally along a substantial portion of the length of the wall.

In another embodiment, the system further includes a plurality of said grooves, whereas each extends circumferentially around a long axis of the filter wall.

In still another embodiment, the groove comprises a helical shape along a length and circumference of the filter wall.

According to another mode of the foregoing system aspects of the invention, the filter wall includes a composite structure with a polymer membrane in combination with a network of structural support struts. The network of structural support struts is coupled to the membrane. A plurality of apertures communicate through the membrane. At least one of the structural support struts spans across each of the the apertures.

According to one embodiment of this mode, the network of structural support struts comprises a plurality of metallic filaments. In another embodiment, the network of structural support struts comprises a metal braid. In another embodiment, the network of structural support struts comprises a plurality of metallic wires. In another embodiment, the network of structural support struts comprises a plurality of metallic ribbons.

According to another mode applicable variously across the system aspects described hereunder, the system further provides a proximal filter assembly with an aspiration catheter and that is adapted to be fluidically coupled to the distal embolic filter assembly at the distal location and to reverse flow at the distal location so as to aspirate contents captured on an upstream side of the embolic filter and to remove said contents from the patient.

According to one embodiment of this mode, the aspiration catheter further comprises an inflatable balloon.

In still another mode of the various system aspects of the invention, the filter wall comprises a surface that is exposed to the blood at the distal location, whereas a bioactive agent is coupled to the surface in a manner expressing substantial bioactivity with respect to the blood in contact with the surface.

In one embodiment, the surface is located on an upstream side of the distal embolic filter.

In another embodiment, the surface is located on a downstream side of the distal embolic filter.

In another embodiment, the surface includes a drug eluting matrix carrier that is different than the bioactive agent and that holds and elutes the bioactive agent. According to one further embodiment, the drug eluting matrix carrier comprises a polymer. According to another further embodiment, the drug eluting matrix carrier comprises a hydrogel. In still another further embodiment, the drug eluting matrix carrier comprises a saccharide.

In still another embodiment, the drug eluting matrix carrier comprises a metal matrix, which may be in particular highly beneficial modes an electrolessly deposited metal matrix, such as in the form of a composite deposited matrix with the bioactive agent.

According to another embodiment, the bioactive agent comprises an anti-platelet adhesion agent. In one more specific embodiment considered highly beneficial, the bioactive agent comprises clopidogrel.

According to another embodiment, the bioactive agent comprises an anti-thrombogenic agent.

In certain more specific embodiments, the bioactive agent comprises at least one of heparin, hirudin, clopidogrel, TPA, urokinase, streptokinase, fluorouracil, abciximab, or IIb/IIIa inhibitor, or an analog, derivative, precurosor, or blend thereof.

According to another mode hereof, the surface comprises a circumferential area that is adapted to engage a wall of the vessel at the location. The bioactive agent comprises at least one of an anti-restenosis or an anti-inflammatory compound. According to one embodiment, the bioactive agent comprises at least one of sirolimus, tacrolimus, everolimus, ABT-578, paclitaxel, Beta-estradiol, nitric oxide (NO), an NO agonist, a statin, dexamethazone, or aspirin.

According to one further mode, first and second bioactive surfaces are provided on upstream and downstream sides of the filter wall, respectively, and have first and second different respective biocompatibilities, e.g. such as for example eluting different agents.

Also included as additional aspects hereof are various methods.

According to one such aspect, a method is provided for forming an embolic filter assembly as follows. A polymer membrane constructed from a first material is masked with a second material that is substantially different than the first material. A bi-layer composite wall is thus formed with a first side corresponding with a first layer constructed principally of the first material and a second side corresponding with a second layer of the second material. The second material is deposited upon the first material with a pattern having a plurality of voids through which portions of the polymer membrane of the first layer are exposed to the second side. The second side is exposed to an ablation source that selectively ablates the first material and not the second material. The exposed portions of the first material are selectively ablated without substantially ablating the second material, and a plurality of engineered pores are formed through the first and second materials and corresponding with the voids in the second material. A distal embolic filter assembly is formed at least in part with the composite wall with engineered porosity from the selective pore ablation.

Another aspect includes a method for manufacturing an embolic filter system as follows. A delivery member with an elongate body is provided. A substantially porous wall is mounted on a super-elastic, nickel-titanium frame that is secured to the elongated body. The frame is provided with a material shape memory in a radially expanded condition, such that the frame is self-expandable from a radially collapsed condition to a radially expanded condition. The frame is held in radial confinement in the radially collapsed condition by at least one releasable circumferential tether that holds the frame substantially tight around the elongated body of the delivery member. The tether is released at the distal filtering location to thereby remove the frame from radial confinement and allow the frame to self-expand to the radially expanded condition.

Another aspect of the invention is a method for manufacturing an embolic filter system as follows. A plurality of discrete apertures are formed through a distal embolic filter wall such that a length of each aperture is at least about twice the width of the respective aperture, and furthermore wherein the width is equal to or less than about 120 microns.

Another method aspect includes a method for manufacturing an embolic filter system as follows. A network of structural support struts is couled to a membrane constructed from a polymer matrix to thereby form a composite structure. A plurality of apertures are formed that communicate through the membrane and such that at least one of the structural support struts spans across each of the the apertures. The composite structure with apertures formed therethrough is used as a wall for a distal embolic filter assembly.

Another aspect according to the invention includes method for manufacturing an embolic filter system, and/or for performing a distal embolic filtering procedured, by providing and using both a distal embolic filter assembly and a proximal embolic filter assembly. A distal embolic filter procedure is conducted at a distal location within a blood vessel in a patient using the distal embolic filter assembly such that antegrade flow perfuses through a substantially porous wall of the distal embolic filter assembly but further such that material is captured at an upstream side of the filter wall. In combination, a proximal embolic filter procedure is conducted on the patient by using the proximal embolic filter assembly to reverse flow at the distal location. In this manner, the material captured at the upstream side of the filter wall is flushed proximally into an aspiration lumen and sheath at a proximal location associated with the vessel.

Another aspect includes a method for performing a distal embolic filter procedure that includes coupling a bioactive agent to a surface of a distal embolic filter wall, wherein the bioactive agent is a different material than the polymeric membrane, and thereby expressing substantial bioactivity with respect to blood in contact with the surface using the bioactive agent.

Another aspect is a method for manufacturing a distal embolic filter as follows. A composite wall is formed with a first layer on a first side comprising a membrane constructed from a first material, and also with a second layer comprising a second material deposited onto the first material. At least one of the first and second materials not inherently porous to the extent sufficient to provide the substantial porosity for embolic filtering and substantially uncompromised blood flow therethrough. A pattern of perfusion pores is formed through the first and second materials. The first and second materials are characterized as being substantially different such that the first material if exposed within the pores to an ablation source would ablate, but whereas the same exposure is not ablative to the second material.

These various aspects, modes, embodiments, variations, and features described above are also further considered within an embolic system wherein the embolic filter is adapted to be used over a guidewire such that the guidewire is provided independent of, though cooperates with, the filter device. Further such additional, independent aspects, modes, embodiments, variations, and features are provided as follows.

In one aspect, the embolic filter device is adjustable between a first configuration and a second configuration, and also between unlocked and locked conditions with respect to the guidewire. In the first configuration and unlocked condition, the embolic filter device is adapted to be slideably positioned over the guidewire at a position where filtering is desired. The filter device is adapted to be adjusted to the locked condition onto the wire at the position. The filter device is further adapted to be adjusted in-vivo to the second configuration that is adapted to filter emboli from fluids flowing therethrough at a filtering location corresponding to the filter device's locked position along the guidewire.

In one mode, the filter device is adapted to filter emboli from blood. In one embodiment, the device is adapted to be positioned with the guidewire downstream from an intervention site in a carotid artery in a patient and to filter emboli released during the intervention at the intervention site.

In another embodiment, the filter system is adapted to be positioned downstream from an anastomosed arterial or venous graft, and is adapted to filter emboli from blood flowing downstream from the graft, such as during an intervention such as recanalization of the graft.

In another mode, the filter device has a filter assembly secured onto a tubular support member. The tubular support member has a guidewire passageway therethrough and is adjustable between a first configuration and a second configuration. In the first configuration the guidewire passageway has a first inner diameter that is adapted to allow the tubular support member to be moveably engaged over the guidewire for adjustable placement of the filter device along the length of the guidewire. In the second configuration, the guidewire passageway has a second inner diameter that is adapted to engage the guidewire sufficient to lock the filter device onto the guidewire such that the filter device remains on the guidewire during in-vivo use.

In another mode, the filter device adjusts to the second configuration in response to an applied energy. In one embodiment, the filter device is adapted to adjust to the second configuration in response to an applied electrical current to a conductor associated with the filter device. In another embodiment, the filter device is adapted to adjust to the second configuration in response to applied ultrasound energy. In another embodiment, the adjustment is in response to an applied light energy.

In another mode, the filter system includes a control system coupled to the filter device and that is adapted to control the positioning, locking, and radial adjusting of the filter device with respect to a guidewire.

According to one embodiment of this mode, the control system includes a delivery member that is adapted to hold the filter device and advance the filter device over a guidewire to the position where it is desired to be locked. The control system in another embodiment includes a lock member that is adapted to lock the filter device at the position along the guidewire.

In another embodiment, the control system includes a radial adjusting system that is adapted to couple to the filter device and adjust it between the first and second configurations. In one variation of this embodiment, the radial adjusting system includes an outer sheath that is longitudinally moveable over the guidewire between first and second positions, respectively, with respect to the filter device. In the first position, the filter device is radially contained within a passageway of the outer sheath in a radially collapsed condition. In the second position, the filter device is located exteriorly of the passageway and is adapted to expand to a memory state that is a radially expanded condition corresponding to the second configuration. In another variation, a pull wire is coupled to a radial support member.

In another aspect, the invention is an embolic filter system with a filter device that includes a filter assembly with a radial support member coupled to a filter wall. In a radially expanded condition, the radial support member supports at least in part the filter wall in a shape that is adapted to filter blood flowing into the assembly of the radially support member and wall.

In one mode, the filter wall is a sheet of material. In one embodiment, the sheet of material comprises a porous membrane with pores having sufficient size to allow normal physiological blood components to pass therethrough, but to filter larger components such as emboli from passing. In another embodiment, the sheet of material has a plurality of apertures formed therethrough.

In another mode, the filter wall is a meshed network of strand material having spaces between strands of sufficient size to allow normal physiological blood components to pass therethrough, but to filter larger components such as emboli from passing.

The invention in another aspect is an embolic filter system having an embolic filter device coupled to a control system that includes at least one detachable member that is detachable from the embolic filter device when the embolic filter device is positioned at a remote in-vivo location.

In one mode of this aspect, the detachable member is a conductor lead that is adapted to couple energy from an ex-vivo energy source to the embolic filter device at the remote in-vivo location. In one embodiment of this mode, the conductor lead is electrolytically detachable from the filter device upon application of sufficient electrical energy to a sacrificial link between the conductor lead and the filter device.

The invention in another aspect is an embolic filter system with an embolic filter device that includes a filter assembly coupled to a locking member. The locking member is adjustable between an unlocked condition and a locked condition. In the unlocked condition, the filter device is adapted to be advanced over a guidewire to a desired position. In the locked condition, the filter device is substantially locked onto the guidewire at the position.

The invention in another aspect is an embolic filter system with an embolic filter device that includes a filter assembly cooperating with an adjustable member. The adjustable member is adjustable between a first shape and a second shape. In the first shape the adjustable member is allow for passage of a guidewire therethrough. In the second shape, the filter device is adapted to be locked onto the guidewire.

In one mode, the adjustable member has a first inner diameter in the first shape, and a second inner diameter that is smaller than the first inner diameter in the second shape.

In another mode, the adjustable member is formed at least in part from a shape-memory material. In one embodiment, the shape memory material is nickel-titanium alloy. In one variation, the nickel-titanium alloy forms an annular member such as a ring. In a further feature, the ring may have a memory state in the second shape. In a further feature, the ring is adjustable between the first and second shapes at a particular temperature. In a further feature, the temperature is above normal resting body temperature.

In another mode, the adjustable member is adapted to be positioned along the guidewire and has a first outer diameter in the first shape and a second outer diameter in the second shape. The first outer diameter is sufficiently small to slideable clearance between the guidewire at the position of the adjustable member and a guidewire passageway of the filter device. The second outer diameter is larger than the first outer diameter and is sufficient to radially engage the guidewire passageway to thereby lock the filter device onto the guidewire at the position of the adjustable member.

The invention according to another aspect is an embolic filter system with an embolic filter device having a filter assembly cooperating with an annular member that is adjustable between first and second inner diameters. The first inner diameter is greater than an outer diameter of the guidewire. The second inner diameter is less than the outer diameter of the guidewire.

In one mode, the annular member is formed at least in part from a shape-memory material. In one embodiment, the shape memory material is nickel-titanium alloy.

In another mode, the annular member is a ring.

In another mode, the annular member is a coil.

In another mode, the annular member is a tubular member.

In another mode, the annular member comprises a pattern of interconnected struts separated by void areas.

In another mode, the annular member is formed at least in part from a solid tubular member that has a pattern of voids cut therein.

In another mode, the annular member has a memory condition in the second shape. In one embodiment, the annular member is adjustable between the first and second shapes at a transition temperature. In one variation, the transition temperature is above normal resting body temperature. In another variation, the transition temperature is equal to about normal resting body temperature.

The invention according to another aspect is a method for providing an embolic filter system, comprising providing an embolic filter device; placing a distal end portion of a guidewire at a remote in-vivo location within a body of a patient; advancing the filter device over the guidewire in a first configuration and unlocked condition to a position along the distal end portion of the guidewire where filtering is desired; locking the filter device onto the guidewire by adjusting the filter device from the unlocked condition to the locked condition at the position; and adjusting the locked filter device at the position from the first configuration to the second configuration that is adapted to filter emboli from fluid flowing into the filter.

According to one mode of this aspect, the method further includes heating the filter device at the position by coupling the filter device to an energy source located externally from the body; and wherein the heat adjusts the filter device from the unlocked condition to the locked condition. In a further embodiment, the heating includes applying an electrical current to a conductor associated with the filter device, and in one variation the method includes applying an RF current to the conductor. In another embodiment, the heating includes optically coupling light to a conductor associated with the filter that is adapted to heat upon absorbing the light. In another embodiment, the heating includes coupling ultrasound energy to a conductor associated with the filter device that is adapted to heat upon ultrasound absorbance. The ultrasound energy may be produced within the system itself within the body, such as by coupling an ultrasound crystal associated with the filter device with an electrical source externally of the body that is adapted to energize the ultrasound crystal to produce the ultrasound energy.

Another mode of this aspect includes adjusting an adjustable member of the filter device from a first shape to a second shape that correspond with the unlocked and locked conditions, respectively, for the device. In the first shape, there is clearance for the filter device to slideably engage and move over the guidewire. In the second shape, the adjustable member engages the guidewire. In one embodiment the adjusting includes reducing the inner diameter of an annular ring. In another embodiment, the adjusting includes reducing the inner diameter of a longitudinally extending coil or braid.

The invention in another aspect provides an embolic filter as a module that is adapted to be removably engaged onto a guidewire.

The invention in another aspect provides an embolic filter that is adapted to be delivered over an indwelling guidewire, positioned at a location along a distal end portion of the guidewire distal to a site of intervention, and locked onto the guidewire at the location.

The invention according to another aspect provides an embolic filter that is adjustable between radially collapsed and radially expanded conditions on a guidewire positioned at a location distal to an intended invention site.

The invention also includes various aspects that are adaptations of the aspects, modes, embodiments, variations, and features above as a proximal embolic filtering system and method.

Another aspect of the invention is an embolic filter system with a filter assembly and an adjustable lock assembly as follows. The filter assembly has a filter member that is adjustable between a radially collapsed configuration and a radially expanded configuration. The filter assembly is adapted to be locked with the adjustable lock assembly at a selected position along a distal end portion of a guidewire at a location within a lumen in a patient's body, and is adapted to be delivered at least in part with the guidewire to the location in the locked configuration. The filter member is adjustable at the location from the radially collapsed configuration to a radially expanded configuration that spans across a substantial cross-section of the lumen. The filter member in the radially expanded configuration at the location is also adapted to filter components of fluid flowing through the lumen at the location above a predetermined size.

Another aspect of the invention is an embolic filter system with a delivery member that cooperates with a filter assembly as follows. The delivery member has an elongate body having a proximal end portion and a distal end portion. The filter assembly has a filter member that is adjustable between a radially collapsed configuration and a radially expanded configuration. The distal end portion of the delivery member is coupled to the filter assembly and is adapted to at least in part advance the filter assembly in the radially collapsed configuration to a location within a lumen in a body of a patient by manipulating the proximal end portion externally of the patient's body. The filter member is adjustable at the location from the radially collapsed configuration to a radially expanded configuration that spans across a substantial cross-section of the lumen. The filter member in the radially expanded configuration at the location is adapted to filter components of fluid flowing through the lumen at the location above a predetermined size. The distal end portion of the delivery member is detachable from the filter assembly at the location.

Another aspect of the invention is an embolic filter system with a delivery member, a filter assembly, and an adjustable lock assembly as follows. The delivery member has an elongate body having a proximal end portion and a distal end portion. The filter assembly includes a guidewire tracking member, and a filter member coupled to the guidewire tracking member and that is adjustable between a radially collapsed configuration and a radially expanded configuration. The distal end portion of the delivery member is detachably coupled to the guidewire tracking member and is adapted to advance the filter assembly with the filter member in the radially collapsed configuration over the guidewire to the location by manipulating the proximal end portion of the delivery member externally of the patient's body. The filter member is adjustable at the location from the radially collapsed configuration to a radially expanded configuration that spans across a substantial cross-section of the lumen. The filter member in the radially expanded configuration at the location is adapted to filter components of fluid flowing through the lumen at the location above a predetermined size. The adjustable lock assembly is adapted to lock the filter assembly onto the distal end portion of the guidewire at the location, and the delivery member is detachable from the guidewire tracking member at the location.

Another aspect of the invention is an embolic filter system with a delivery assembly that cooperates with a filter assembly as follows. The filter assembly has a filter member having a wall with a substantially annular passageway around a circumference, and with a superelastic loop-shaped member coupled to the filter member within the annular passageway and along the circumference. The superelastic loop-shaped support member is adjustable between a radially collapsed condition corresponding with an elastically deformed condition for the loop-shaped member and a radially expanded condition according to material recovery from the elastically deformed condition to a memory condition. Adjusting the support member from the radially collapsed condition to the radially expanded condition adjusts the filter member between a radially collapsed configuration and a radially expanded configuration, respectively. The filter assembly is adapted to be delivered at least in part with the delivery assembly to a location within a lumen in a body of a patient with the support member radially confined in the radially collapsed condition and the filter member in the radially collapsed configuration. The support member and filter member are adjustable from the radially collapsed condition and radially collapsed configuration, respectively, to the radially expanded configuration and radially expanded configuration, also respectively, at the location. The filter member in the radially expanded configuration at the location spans across a substantial cross-section of the lumen. The filter member in the radially expanded configuration at the location is adapted to filter components of fluid flowing through the lumen at the location above a predetermined size.

Another aspect of the invention is an embolic filter system as follows. The system includes a delivery member with an elongate body having a proximal end portion and a distal end portion with a longitudinal axis, and a lumen extending between proximal and distal ports each being located along the distal end portion. The system also includes a filter assembly with a filter member coupled to a support member and that is adjustable from a radially collapsed configuration corresponding with an elastically deformed condition for the filter member and to a radially expanded configuration according to memory recovery from the elastically deformed condition toward a memory condition. The filter assembly in the radially collapsed configuration is radially confined within the lumen and is adapted to be delivered to a location within a lumen in a body of a patient. The filter assembly is adjustable from the radially collapsed configuration at the location to the radially expanded configuration at the location by removal of the filter assembly from the radially confining lumen. The filter member in the radially expanded configuration at the location spans across a substantial cross-section of the lumen, and is adapted to filter components of fluid flowing through the lumen at the location above a predetermined size.

Another aspect of the invention is a method for filtering emboli from fluid flowing across a location within a body lumen in a patient that includes the following steps. A filter assembly is delivered in a radially collapsed configuration over a guidewire to the location. The filter assembly is locked onto the guidewire at the location, and is then adjusted from the radially collapsed configuration to a radially expanded configuration at the location. The filter assembly in the radially expanded configuration at the location spans across a substantial cross-section of the body lumen and is adapted to filter the emboli from the fluid flowing across the location.

Another aspect of the invention is a method for filtering emboli from fluid flowing across a location within a body lumen in a patient as follows. A filter assembly is delivered with a delivery member in a radially collapsed configuration over a guidewire to the location. The filter assembly is detached from the delivery member at the location. The filter assembly is adjusted from the radially collapsed configuration to a radially expanded configuration at the location, which spans across a substantial cross-section of the body lumen and is adapted to filter the emboli from the fluid flowing across the location. The filter assembly is thereafter collapsed with filtered emboli captured therewith. Then, the collapsed filter assembly is removed from the body lumen.

Another aspect of the invention is another method for filtering emboli from fluid flowing across a location within a body lumen in a patient as follows. A filter assembly is positioned in a radially collapsed configuration within a capture lumen of a radially confining cuff having an adjustable position relative to the filter assembly. The filter assembly is provided in the radially collapsed configuration within the adjustable radially confining cuff along a distal end portion of a delivery member. The distal end portion of the delivery member and filter assembly are delivered in the radially collapsed condition within the cuff to the location, and the filter assembly is adjusted from the radially collapsed configuration to a radially expanded configuration at the location by adjusting the relative position of the cuff relative to the filter assembly such that the filter assembly is released from radial confinement and self-expands according to material memory to the radially expanded condition. The filter assembly in the radially expanded configuration at the location spans across a substantial cross-section of the body lumen and is adapted to filter the emboli from the fluid flowing across the location. The filter assembly is thereafter collapsed with filtered emboli captured therewith by positioning the filter assembly at least in part back within the radially confining cuff, and is removed at least partially confined within the cuff from the body lumen. Further to this method, the capture lumen extends along a length between proximal and distal ports and is located entirely within the body lumen, such as for example when the filter assembly is located within the cuff to the location.

Another aspect of the invention is a method for assembling an embolic filter system as follows. A guidewire is provided that has a proximal end portion and a distal end portion with a first length that is adapted to be positioned at a location within a lumen in a patient while the proximal end portion extends externally from the patient. A filter assembly is also provided with a filter member coupled to a guidewire tracking member having a guidewire lumen extending with a second length between a proximal port and a distal port. The guidewire lumen is slideably engaged over the guidewire. The second length is less than the first length, such that the filter assembly is a shuttle that tracks over the guidewire. The shuttling filter assembly according to a further mode is locked onto the distal end portion of the guidewire.

The various aspects, modes, embodiments, variations, and features just described are to be considered independently beneficial without requiring limitation by the others. However, further combinations and sub-combinations apparent to one of ordinary skill are also contemplated as within the scope of the present invention. Other beneficial aspects, modes, and embodiments are to be appreciated by one of ordinary skill based upon further review of the disclosure below and accompanying Figures.

### BRIEF DESCRIPTION OF THE DRAWINGS

FIG. 1 shows an angular perspective view of a partially cross-sectioned portion of a membrane material in a first condition associated with one mode of preparing an embolic filter membrane having an engineered porosity and bioactive surface according to one embodiment of the invention.

FIG. 2 shows another angular perspective view of the partially cross-sectioned portion of membrane material shown in FIG. 1, although in a second condition associated with another mode of preparing the embolic filter membrane.

FIG. 3 shows another angular perspective view of the partially cross-sectioned portion membrane material shown in FIGS. 1 and 2, although in a second condition associated with another mode of preparing the embolic filter membrane.

FIG. 4 shows another angular perspective view of another partially cross-sectioned portion of membrane material similar to that shown in FIG. 3, except showing a larger portion of the material resulting from the mode shown in FIG. 3 and revealing a dense pattern of engineered pores across a sheet of the engineered membrane.

FIG. 5 shows a plan view of a sheet of porous membrane material cut into a particular pattern adapted for use as a precursor material to form an embolic filter wall according to another embodiment of the invention.

FIG. 6 shows an angular perspective view of the sheet of membrane material shown in FIG. 5, although in a subsequent mode of preparing an embolic filter wall assembly.

FIG. 7 shows a schematic view of a support ring adapted for use with the membrane shown variously in FIGS. 5 and 6 in assembling an embolic filter assembly.

FIG. 8 shows a side view of an embolic filter assembly constructed according to the various modes and components shown in FIGS. 5-7.

FIG. 9 shows a side view of the embolic filter assembly shown in FIG. 8 during one mode of combination use with a guidewire.

FIG. 10 shows an angular perspective view of a cross-sectioned portion of composite membrane material adapted for use in preparing an embolic filter assembly according to another embodiment of the invention.

FIG. 11A shows an angular perspective view of a sheet of composite membrane material similar to that shown in FIG. 10, except in larger scale and cut into a pattern adapted for use in preparing an embolic filter assembly for use in a patient.

FIG. 11B shows an exploded view of a perfusion groove that includes certain bridging support struts according to one feature appropriate for use in the embodiment shown in FIGS. 10-11A.

FIG. 12 shows an angular perspective view of the cut sheet of membrane material shown in FIG. 11A, except in subsequent mode of preparing the embolic filter assembly for endolumenal use in a patient.

FIGS. 13A-B show two alternative patterns of grooved perfusion configurations for an embolic filter assembly according to additional embodiments of the invention.

FIGS. 14A-B show schematic side views of one form of a detachable tether assembly that is adapted to adjust a filter assembly according to one or more of the foregoing embodiments from a radially collapsed configuration to a radially expanded configuration without the need for axial withdrawal of a coaxial delivery sheath.

FIG. 15 shows a side view of a distal end portion of a distal embolic filter assembly in a radially collapsed condition for delivery and according to use of the tether assembly similar to that shown in FIGS. 14A-B.

FIG. 16 shows a side view of a similar distal embolic filter assembly to that shown in FIG. 15, except following release from radial confinement and upon opening for filtering use in-situ in a patient.

FIG. 17 shows a side view of a similar distal embolic filter assembly to that shown in FIG. 16, except following a filtering procedure and upon use of a radial capture sheath to collapse the assembly down for removal from a patient.

FIG. 18 shows a schematic view of a combination filtering assembly that includes a distal embolic filter assembly similar to that shown in FIG. 16 during one mode of use in a distal embolic filtering procedure in a vesse, and a proximal embolic filter assembly during one mode of use to flush or clear the distal filter that remains in a filtering position in the patient.

### DETAILED DESCRIPTION OF THE EMBODIMENTS

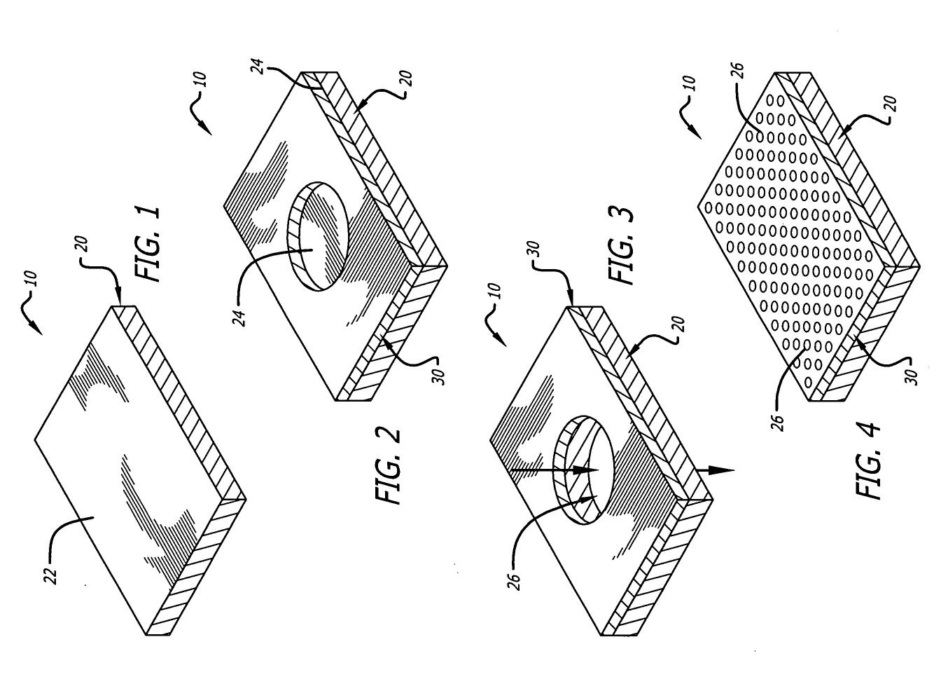

FIG. 1 to FIG. 18 show various modes of operation in preparing a distal embolic filter assembly, and various other embodiments and modes of use, according to various aspects of the present invention as follows.

FIG. 1 shows an illustrative portion of an initial form of a filter wall 10 that includes only a sheet of membrane material 20 that does not, at this stage, have an inherent porosity that is a desired porosity for embolic filtering. Nor does it provide all the surface features desired in an ultimate surface according to various of the present embodiments. However, it provides other desirable features as a wall material for use, and is used as a precursor material for preparation of the engineered material of desired porosity. Membrane 20 includes a top surface 22 that provides a platform upon which another second material will be deposited in order to achieve certain objectives of the embodiments described below.

For many materials and methods, patterned ablation tools and techniques may be available to process a starting material such as membrane 20 in order to etch or photoablate, etc., a patterned porosity of desired parameters therethrough. However, in many other cases, the desired wall material may not be a suitable material for such selective material processing. This is may be in particular the case for micro-scale processing of dense patterns and shapes of structures or surface morphologies, such as for example in the case of micro-porous blood filters. In certain such cases therefore, more intensive engineered processes may be employed to achieve the desired engineered result. In many such processes, additional surface materials are added, either as a permanent part of the structure, or in other circumstances as sacrificial materials along a process, e.g. to provide masking or other assistance to the desired selective material removal or processing.

In the present embodiments, a modified surface is provided that assists both in selective masking for photoablation or chemical etching of an engineered porosity (e.g. post-processing of a starting non-porous membrane 20), as well as for enhanced surface characteristics of the ultimately intended device.

FIG. 2 shows a subsequent stage of operation wherein a second sheet of material 30 is deposited, laminated, coated, or otherwise laid down or formed upon surface 22 of membrane 20. More specifically, as shown in FIG. 2, material 30 is deposited in a manner leaving a void 32 therethrough that results in a pattern or shape 24 where surface 22 of underlying membrane 20 is not covered. Material 30 is different than the material make up of membrane 20 in such a way that an applied energy or chemical does not affect the portions of membrane 20 covered by material 30. In this manner, material 30 acts as a shield. However, the applied energy or chemical selectively ablates the exposed portion(s) 24 where material 30 is missing. This is shown by schematic arrows in illustrative form in FIG. 3, and results in the formation of a pore 26 of engineered size and shape based upon the selectively patterned coating of material 30 upon membrane 20.

As shown in FIG. 4, a resulting pattern of such pores 26 results when this process is done over a larger area and according to a pattern of such voids of uncoated portions of membrane 20 that are unshielded by material 30 and thus ablated. This pattern of engineered may be created together, and do not require discrete drilling etc. of holes through the material. The actual sizes, shape, distribution and density of the pores may vary according to an extremely wide degree of freedom and to meet a variety of needs. Patterned etching techniques are well known in many industries, including for example stent cutting as well as silicone wafer and integrated circuit patterning and manufacturing etc. Pattern light activitation may for example change the chemical make-up only where exposed to certain wavelength and intensity of applied light, or in the presence of certain materials to react at the surface 22. This patterned reaction may for example lay the groundwork pattern for deposition of material 30 in yet a further reaction.

The surface 22 may be activated for selective deposition of material 30 in the pattern shown in FIG. 2, or on the wider scale as represented by FIG. 4 in more advanced form of material processing according to that pattern. Or, the deposition of the material 30 may be selectively patterned on its own, such as via activated etching and reaction processes similar to that just described.

Or, another specific stepwise method of selectively surface coating and selectively ablating patterned pores (not shown) is also further contemplated as follows. A material deposition is first selectively formed at the areas where pores are desired. For example, a first sacrificial material may be laid down in a pattern of separated circular areas with a certain thickness on surface 22 (eg. bumps of coated material). These are intended to correspond with where the pores will go, and generally their intended size, shape, etc. This first material is a sacrificial masking material. Then, material 30 is deposited as the second material that selectively deposits around the masked areas as they are chosen to be unreactive with the surface process allowing for the deposition of material 30. Then, the sacrificial masking material is removed.

Still further, additional steps may also be taken to achieve the desired result. In this regard, a first sacrificial material forms the pattern of the second layer with voids where the pores are to be formed. A second sacrificial material is deposited in the void areas of the first sacrificial material surface layer. The first sacrificial material is removed. Then material 30 is laid down selectively around the second sacrificial material in the intended pore pattern. The second sacrificial material is ablated.

Yet a further approach may include laying material 30 down uniformly over membrane surface 22. Then, voids in material 30 (e.g. as shown in FIG. 2) are selectively ablated. Then, the exposed portions 24 of material 20 are ablated through the voids formed in material 30. This process may be used for example where there is not a good selective source for engineering a patterned ablation to the material composition of membrane 20, but there is a selective ablation technology and patterned ablation technology of material 30. For example, selective ablation of material 30 to form the voids therein may be done with one wavelength of patterned light that ablates that material 30 but not the material of membrane 20. Then, another wavelength of light may be used to expose the whole surface and that ablates the material of membrane 20 but not material 30—however, membrane 20 is photo ablated only where the exposed areas 24 see the light.

In various of these methods and techniques noted above, material 30 may remain a part of the ultimate filter membrane used for medical procedures as a product. Or, the material 30 may then thereafter be removed as a sacrificial material used in order to achieve the engineered porosity of the underlying material of membrane 20. In many cases, however, material 30 may present substantial further beneficial aspects to improved devices and methods, as will be further developed below.

In one particular highly beneficial mode, electroless plating deposition may be used for selective surface coating or masking as just described, including for deposition upon polymer membrane. Such may use for example selective activation upon the surface of the polymer membrane 20 for selective electroless deposition as material 30. In general, electroless deposition includes a metallic material in combination with a reducing agent of that metal, e.g. most typically nickel and phosphorous. An electroless bath is prepared that provides the environment for spontaneous, autocatalytic co-deposition of these two materials which is an oxidation/reduction procuess from their ionic form in the bath and into nano-granular condensed solid (but often somewhat nano- or micro-porous) matrix on the activated surface exposed to the bath. In general, an “activated” surface is typically an electrically conductive surface such as nickel alloy etc. that may provide a substrate for an exchange of electron charge in an atomic circuit of the oxidation/reduction process. Polymers and other materials have been rendered activated for electroless deposition using various previously disclosed methods. In one regard, a combination of stannous chloride bath activation, followed by a palladium bath step, provides a stannous-palladium “nucleated” surface with various “nucleation” sites upon which the electroless deposition may autocatalyze and begin growing. For example, glass is frequently coated with electroless nickel-phosphorous in this manner. Polymeric balloon materials have also been disclosed for electroless nickel deposition, such as for example in order to carry radioactive charge for vascular wall therapy.

Composite deposition of particulate materials within metallic matrixes is also possible using electroless deposition to create metallic surface composites. In general, these materials are typically substantially insoluble but suspended particulate within an electroless bath that is captured in the oxidated/reduced metal/phosphorous surface as impurity. Material particulate such as diamond, polytetrafluoroethylene (PTFE, e.g. or Teflon™), or silicon carbide have been used in composite deposition of engineered surface coatings in this manner. In addition, certain prior disclosures have also described local drug delivery applications for composite deposition of drugs on medical device surfaces (and other techniques using electroless or electroplating deposition of surfaces for local drug delivery of drugs).

Additional examples of electroless deposition are disclosed in the following published PCT International Patent Application: WO 03/045582 to Gertner et al. Additional disclosure is found in the following published U.S. Patent Application: US 2003/0060873 to Gertner et al. The disclosures of these references are herein incorporated in their entirety by reference thereto.

Additional examples of electroless deposition are disclosed in one or more of the following publications: Gertner, Michael E. et al., Drug Delivery from Electrochemically Deposited Thin Metal Films, Electyrochemical and Solid-State Letters, 6(4) J4-J6 (2003); and Gertner, Michael E., et al., Electrochemistry and Medical Devices Fried or Foe?, The Electrochemical Society Interface, Fall 2003. The disclosures of these references are herein incorporated in their entirety by reference thereto.

Use of electroless deposition for material layer 30 provides multiple benefits. In one regard, electroless surfaces have been suggested to improve biocompatiblility of underlying polymer substrates. In another regard, a patterned metallic surface is in particular different from most polymer substrates in a way that readily allows for selective photo, e.g. laser, ablation of the voided metallic regions and exposed polymer substrate. Certain wavelengths of light are known to ablate polymers and have little if no effect on a metallic substrate. By patterning voids in the metal layer and exposing the whole composite layered substrate to such light, the pattern of pores as desired results in a highly robust and scalable process.

In still another very beneficial regard, use of electroless deposited layer for material 30 may include composite deposition or otherwise loading of drugs or other active agents into the metal matrix surface. This provides substantial benefit for local elution of such agents at the surface. In this regard, electroless deposition of material 30 even onto a pre-formed underlying substrate of desired porosity provides substantial benefit for improved filter materials. This applies even without requiring the other added possible benefit of using the metal matrix in a patterned way on a more solid substrate membrane to allow for selective ablation to form the desired porosity.

It is to be appreciated therefore that other materials may be used for material 30 to achieve local drug elution or otherwise engineered bioactivity at the filter membrane surface. Other coating technologies that may be suitable include one or more layers of polymer drug carrier vehicles, such as for example similar to those being used in commercially available or otherwise published technologies for drug eluting stents. Such may be permanent material layers, or erodable or degradable carrier materials. Materials such as for example PEG, PLLA, or PLGA that are well known local drug delivery carriers may be used. Other materials such as hydrogels, saccharides, etc. may also be used as drug carriers. Or, the coating itself may provide some enhanced bioactivity for a particular intended purpose.

In general, various benefits may be provided with improved local bioactivity on embolic filter surfaces according to the various aspects of the invention. In one particular regard, thromboresistance is a substantial benefit that may be provided with anti-platelet adhesion, anti-thromin, or thrombolytic agents either held on the surface or eluted therefrom. This may for example benefit prevention of thrombus formation on the filter surface itself as a foreign body in the blood pool. In combination or alternative to this benefit, elution of such agents locally from the filter provides substantial benefit to lyse or prevent clot downstream from the filter (e.g. due to compromised hemodynamics through and around the filter). Still further, lytic compounds may substantially benefit the filtering process by debulking thromboemboli contents successfully caught by and contained within or on the filter.

Further more detailed examples of these types of agents contemplated hereunder may include for example: clopidogrel (e.g. Plavix™), heparin, hirudin, IIb/IIIa inhibitors, abciximab, TPA™, urokinase, streptokinase, or the like.

Other agents that may be beneficially held on or eluted from embolic filters according to further embodiments include other dissolving agents for debulking of other types of filter contents, such as agents that dissolve calcium, lipids, or cholesterol for example. Statins are one class of compounds that may be used.

One or more of these types of compounds or agents may be held and/or eluted from the bioactive surface described. For example, agents such as certain forms of heparin or other materials such as endothelialization factors (e.g. antibodies similar to used by Orbis™ Corporation for deposition of endothelial progenitor cells on stents) maybe held in a manner on the filter surface to achieve the intended bioactive result (e.g. thromboresistance in the first case, and endothelialization in the latter case such as for longer term indwelling filters such as vena cava filters). The various combinations thereof these various types of agents, either held on the surface and/or eluted therefrom, are also contemplated as would be apparent to one of ordinary skill are also contemplated for more complex and beneficial combined results.

Returning to the Figures, further use of the embodiments described by reference to FIGS. 1-4 is described for final assembly of an embolic filter assembly as follows.

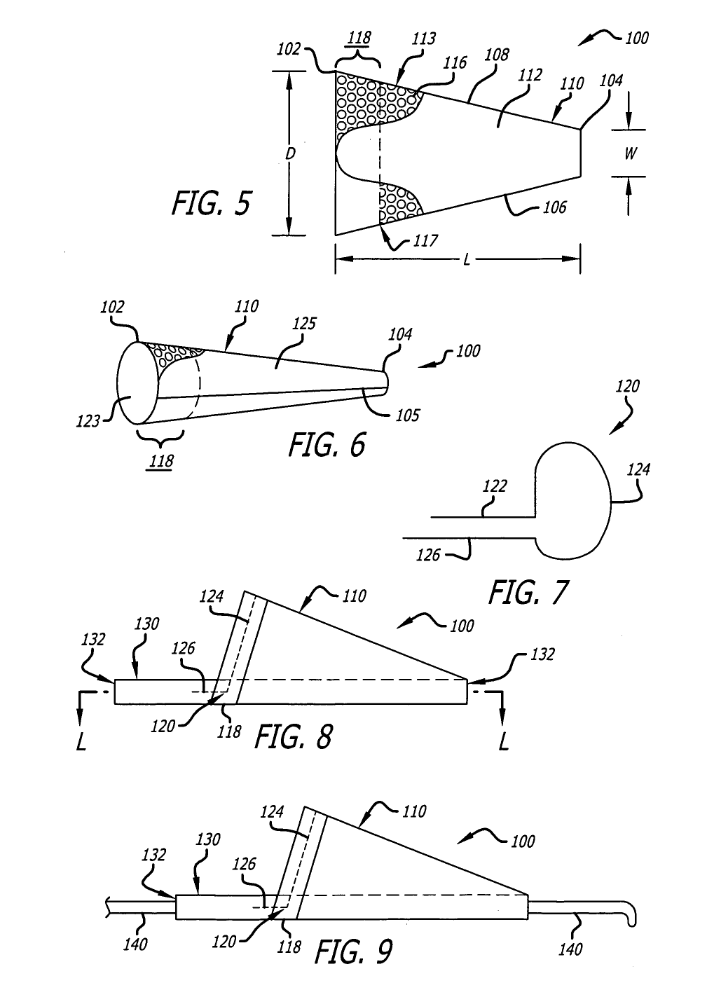

As shown in FIG. 5, a porous filter wall 100 is provided in a precursor configuration that includes a porous membrane 110 in the patterned shape shown. Such pattern may be achieved for example by cutting the pattern from a sheet, or the membrane 110 may be formed in this shape to begin with. Moreover, the pattern may be provided either before or after forming the desired porosity in the material, e.g. shown at pores 116 in partial cut-away view. In one particular beneficial illustrative embodiment, a sheet of material such as shown and described for FIGS. 1-4 is formed, from which multiple patterned pieces such as shown in FIG. 5 are cut. In any event, membrane 110 tapers over a length L between a relatively larger width or diameter portion W at proximal end 102 (e.g. transverse to a longitudinal axis 1), to a relatively smaller width or diameter portion w at distal end 104.

The pattern for filter wall membrane 100 shown in FIG. 5 allows formation of a tapering tubular or frustro-conical shape shown in FIG. 6. In this stage of assembly, filter wall membrane 100 tapers from proximal end 102 having a first diameter D to distal end 104 having a relatively smaller second diameter d. This is formed by securing the lateral edges 106,108 of membrane 110 together along length L, such as along fused line 105 shown in FIG. 6.

A radial support ring 120 is shown in FIG. 7 in partial schematic view, and is incorporated into the filter assembly in conjunction with the filter wall membrane 110 as follows (and by further reference variably to FIGS. 5-8). Ring 120 includes two opposite end portions 122,126 that extend along side of each other from a partial loop portion 124. Loop portion 124 is placed within a circumferential pouch region 116 formed in filter wall 100, such as by inverting or everting edge 102 over upon membrane 110 and securing it in that configuration. This may be done for example by use of adhesives or melt bonding the two confronting portions of similar membrane material to itself (or otherwise mechanically affixing such as by stitching). Ring 120 may be inserted into the pouch region either before formation of the pouch, such as at either of the stages of assembly shown in FIG. 5 or 6 (e.g. flat configuration requiring deflection of loop portion 124, or in the formed tapered tubular configuration shown in FIG. 6). In other words, the pouch may be formed at pouch region 118 by everting or inverting the wall around the loop portion 124 of ring 120. Or, the pouch 118 may be first formed, and loop portion 124 may be inserted into the pouch through apertures or other entry points provided into the pouch. In either case, once ring 120 is so positioned within the pouch, it provides a radial support for filter wall membrane 110 such that filter wall 100 may be converted between open and closed configurations by radial expansion or compression of ring 120, respectively.

In general, ring 120 is constructed from a substantially elastic material or otherwise shape memory material allowing material shape recovery properties to self-expand the ring to the open configuration shown in FIGS. 7-9 (the closed configuration is held under applied radial retention forces). In one particular embodiment, ring 120 is a nickel-titanium alloy well suited for this purpose and intended use in the assembly.

It is to be appreciated that other additional rings may be used as support structures for the filter wall assembly, either along the length or at the opposite end, or both.

Ends 122,126 of ring 120 provide a coupling assembly that assists in securing the support ring 120 to a spine or base 130, as shown in FIG. 8 as a guidewire tracking member or tube. Spine 130 is shown as an elongate tubular member with a lumen 132 that is adapted to slideably receive and track over a guidewire. This may be done as one long assembly with a proximal end portion extending from the patient, or as a shuttle device as shown in FIG. 9 that becomes a part of a guidewire 130 in-situ after being positioned over that guidewire.

It is to be appreciated that further assembly techniques and arrangements may be included in forming the overall filter wall assembly 100 shown in completed form in FIG. 8. For example, further securement of the filter wall membrane 110 to base 130 may include incorporation of the base 130 during the formation stage of the conical structure shown in FIG. 6.

Returning however to illustrative FIG. 6 to further develop additional aspects hereunder, it is also to be appreciated that the local drug delivery or otherwise surface modifications for local bioactivity of the filter membrane 110 may be provided at various locations along the assembly.

In one regard, the bioactivity may be provided on the downstream surface 125 of the filter, which is the outer radial surface in the tubular formed in FIG. 6, 8, or 9. In this particular embodiment, surface 112 shown in FIG. 5 may include a material such as material 30 in FIGS. 2-4 that carries and elutes bioactive agent. In this case where the location of the bioactive surface 112 as the top surface in FIG. 5, the tubing shown in FIG. 6 is formed by confronting edges 106,108 downward into the page such that surface 112 of the FIG. 5 mode remains the outer surface 125 according to the FIG. 6 mode. In any event, much benefit may be provided with certain particular bioactivity provided on this surface.

In particular, local surface activity or agent elution here of anti-platelet or anti-thrombin, or thrombolytic, agents has beneficial impact on thrombus formation at or distally beyond the back side of the filter membrane where platelet may adhere and thrombus may form due to disrupted eddy flow currents and low pressures here distally adjacent the flow ports or pores. In one particular embodiment, heparin is attached to this back surface 125, such as similarly described for “HEPACOTE™” that has been investigated on implantable stents by Johnson & Johnson Corporation, Cordis Division. In another particular embodiment, lytics or other preventative agents such as anti-platelet or anti-thrombus agents are eluted from this surface, such as from a drug eluting polymer carrier surface or electroless deposited composite surface here.

In another regard, bioactivity may be provided along surface 123 shown in FIG. 6 within the radial confines of tubular shaped filter wall 100. In this case, the surface 112 shown in FIG. 5 would become the inner surface of the tube to the extent that is the surface carrying the bioactive agent such as via material 30 in FIGS. 2-4. Here, similar benefits are provided as may be provide on the back surface 125. However, in addition, other materials such as lipid or calcium dissolving agents may be beneficially eluted to debulk contents of the filter within its radial confines defined by surface 123.

The various combinations of bioactivities across these various filter wall surfaces, and various combinations of bioactivities, as described herein are also contemplated. It is also to be appreciated that surface characteristics, or other wall characteristics, may vary in other ways along filter wall 100, such as in particular for example along its length L. One particular example is shown in FIG. 5 by comparison between partially cut-away portions 113 and 117 of membrane 110. More specifically, portion 113 shows porosity everywhere as one continuous material with uniform characteristics, including where pouch region 118 is indicated for later processing to form the retention pouch for support ring 120. However, portion 117 shows a more selective membrane construction, wherein porosity is not provided through the membrane 110 along the region 118 where the pouch for ring 120 is to be formed. In this embodiment, the porosity engineered for passing blood flow does not provide for this, as it is modified in assembly to the retention pouch structure shown in FIG. 8. Thus the pores provide little or no benefit to flow, and may in fact be pro-thrombogenic due to the ingress of blood there through and substantial stasis that may result within and around the porous pouch 118 and retained ring 120.

Also, unique respective bioactivity may be provided at unique locations. In one particular example, the pouch region 118 is generally adapted to circumferentially engage a vessel wall to anchor and support the filter assembly 100 during use. Thus, to the extent there may be harmful damage done to the endothelium or other tissues there, local drug elution may be customized to this area. In one specific example, anti-inflammatory compounds such as aspirin or dexamethasone may be eluted along this pouch 118 when engaged with a vessel wall. In another specific example, other anti-restenosis agents may be eluted. Agents such as sirolimus, tacrolimus, paclitaxel, beta-estradiol, ABT-578, everolimus, statins, nitric oxide, nitric oxide agonists, or analogs or derivatives, pro-drugs, or combinations thereof are contemplated as further examples.

Various further embodiments are described as follows, and may be taken in combination with or separate from the prior embodiments above.

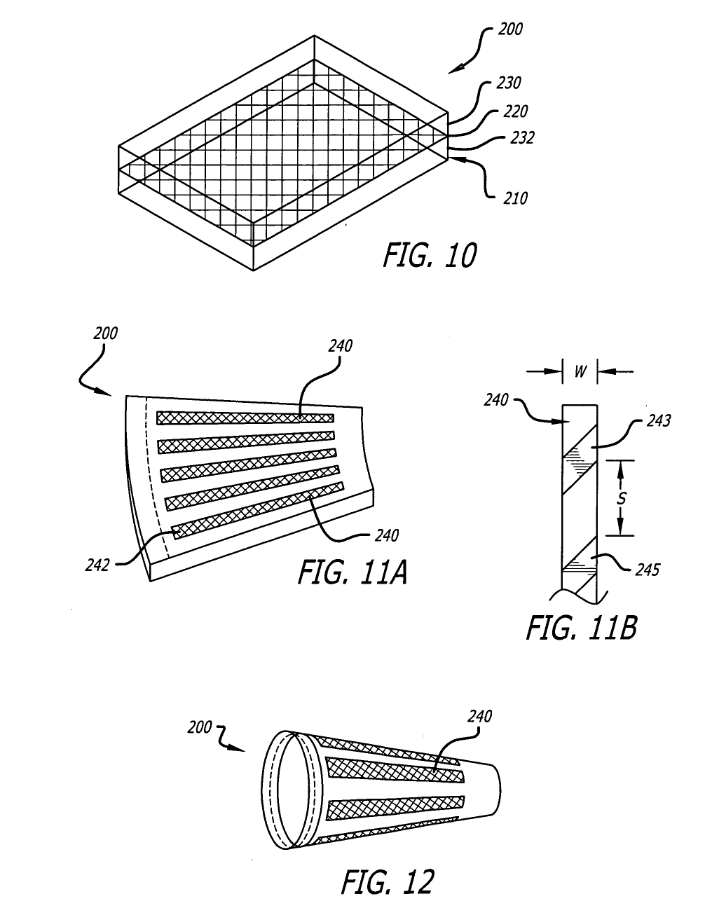

FIGS. 10-12 in particular show various details and views of another filter assembly 200 according to the invention in various modes of assembly as follows. Filter assembly is shown in FIG. 10 in a first precursor form that includes only a composite filter wall 210 prior to being processed into a filter assembly with engineered porosity, and is similar to that depiction in FIG. 1 or 2 for prior embodiments. FIG. 11A shows a patterned piece of wall material 210 in a later mode of assembly and is similar in stage and shape, though with substantially varied wall construction, as that embodiment shown in FIG. 5. FIG. 12 shows a similar view and stage of construction as that shown in FIG. 6 for the prior embodiment. These similar views share similar features and aspects where appropriate, with differences that are clearly found in the following.

More specifically, the present embodiment includes a composite wall construction that includes a composite filter wall 210 constructed from a braided network of metallic fibers 220 embedded within a polymer matrix 230. Polymer matrix 230 may be one material, in which case the assembly may be made by either laminating layered technique with bonding between top and bottom layers (e.g. see area designated for material 230 above braid 220 and area below braid 220 designated as layer 232). Or, such may be accomplished via dipping, spray coating, etc. techniques onto braid 220. In still another further embodiment, braid 220 may be co-extruded through a die with polymer matrix 230 formed thereover. Alternatively, layer 232 may be a different material than the top layer, e.g. where certain bioactivity is desired on one side of the filter and not the other. This also may be accomplished via various of these types of techniques noted above, or otherwise according to one of ordinary skill upon review of the current disclosure and other available information. It is also to be appreciated that the composite nature of the materials do not require completely intermediate positioning of the braid 220 within the polymer matrix 230 as shown in FIG. 10. Rather, this may vary, and in fact braid 220 may be otherwise secured to the polymer matrix 230 without embedding the same, e.g. as laminate materials, as further contemplated embodiments hereof.

One particular series of further more detailed examples of composite materials with laminated layers of varied porosity is disclosed in the following published PCT Patent Application, which is herein incorporated in its entirety by reference thereto: WO 2004/082532 to Kreidler et al. and assigned to ev3, Inc.

As described for other illustrative embodiments above, according to the present embodiments providing a braid re-inforced polymer wall for embolic filter construction and assembly, selective photoablation such as via certain light sources (e.g. certain laser wavelengths) removes the polymer, but not the metal. As such, longitudinal grooves 240 are made possible for less thromboembolic or hemodynamically compromised flow therethrough than previous discrete rounded pore embodiments for embolic filters.

More specifically, typical porosity sizes in other prior filter devices generally range from between about 60 microns to about 120 microns, and still more typically between about 80 microns to about 100 microns, with most efforts settling around 100 micron pores. This is because 120 micron pores are generally believed to let too much emboli through, whereas 60 to 80 micron pores carry concerns regarding hemodynamic compromise and hemolysis and thrombogenicity. Moreover, by providing longitudinal grooves such as shown in FIGS. 11 and 12 according to more conventional filter wall materials, such would compromise the wall integrity around these grooves or cuts therethrough.

In contrast, according to the current embodiment of the invention, the grooves 240 include bridging struts 242 of the braided support structure, which may be for example greater than 100 microns apart, and even greater than 120 microns apart, and still further may even be 200 or even several hundred more microns apart along the long axis L (whereas the groove dimension transverse to the long axis L may be instead for example between about 60 to about 100 microns, and may be even smaller. Such dimensions still do not compromise either the wall integrity or the hemodynamic integrity, as the lateral dimension may be defining for embolic filtering and may be for example within the typical ranges noted above, or even lower due to the benefits of significant growth of the passages in the other longitudinal dimension. Again, by conventional techniques, providing the related braid structure component 220 alone would not be appropriate as the wide spacing would not suffice for the desired porosity of the wall for embolic filtering. Similarly, providing the polymer matrix component 230 alone also would not be appropriate as it would lose its integrity to a dramatic extent without the accompanying bridging structure of the braid across the grooved gaps 240. Thus, only by providing the wire reinforced polymer composite wall with selective patterned perfusion grooves may the present embodiment be most appropriately achieved.

For further illustration, FIG. 11B shows one illustrative portion of a groove 240 wherein two adjacent bridging support struts 243,245 are separated by a distance S that is more than double, and in fact more than three times, the width w for the particular illustrative groove shown. It is believed that, at appropriate specific dimensions for a specific case, this arrangement provides superior combination of hemodynamics and filtering capability versus a comparison structure that would be possible with simple pores of either diameter w or S.

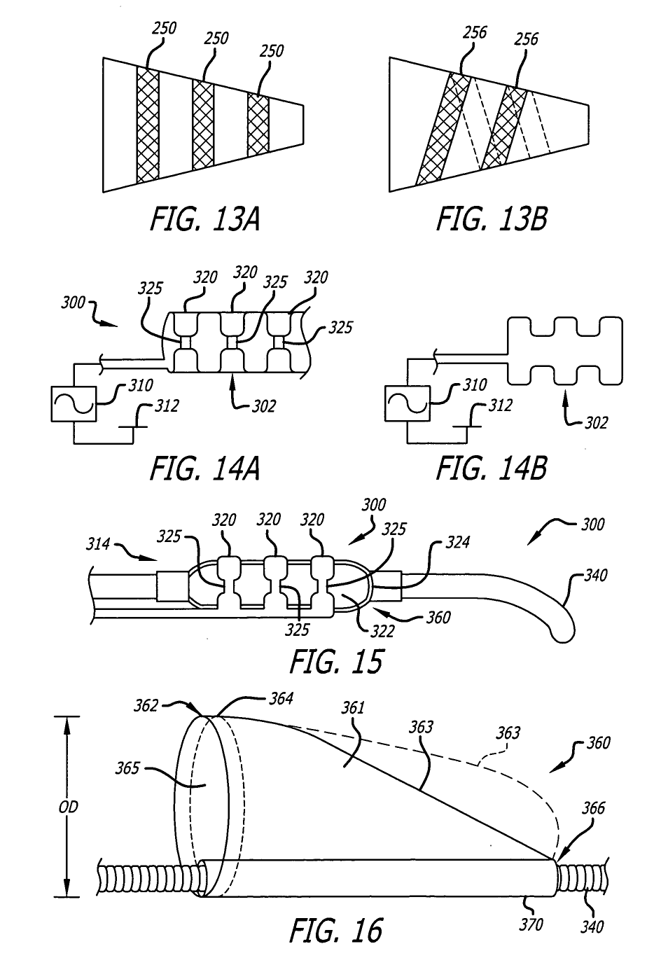

It should be appreciated that, despite specific benefits afforded by the present detailed embodiments, other specific structures are also contemplated. This includes, for example, structures other than specifically braided support struts, such as for example a coiled structure or other network of fibers or support strut members sufficient to provide reinforcement across relatively long cut grooves through the plastic to hold and retain their dimension and form during use. Moreover, other patterns than longitudinal grooves may be formed through such composite. This includes for example circumferential grooves 250, helical grooves 256, etc., as schematically shown in FIGS. 13A-B, respectively, for further illustration.

According to a further embodiment illustrated by reference to FIGS. 14A-17, an embolic filter system 300 includes a filter assembly 360 with a super-elastic nickel-titanium frame 364 that is secured to an elongated body of a delivery member 370. The frame 364 has a memory in the radially expanded condition, and is self-expanding from the radially collapsed condition to the radially expanded condition. The frame 364 is held in radial confinement in the radially collapsed condition by use of a retension assembly 302 that includes one or more releasable circumferential tethers 320 that hold the frame 364 tight in collapsed condition around the elongated body of the delivery member 370. The tether 320 is released at the distal location to thus remove the radial confinement and allow the frame 364 to self-expand to the radially expanded condition. Further refined modes of this aspect include the following.

In one regard, the tether 320 is a wire that is coupled around the frame in a manner to hold it taught in a first mode, but has a sacrificial link 325 which in a second mode is deformed/dissolved/degraded/broken by use of applied energy, such as electrical, optical, etc. in order to release the tether 320.

In one mode, an electrolytic process is used similar to that used to detach embolic coils for treating neuroaneurysms, and otherwise as previously described for other detachable medical devices. This provides substantial benefits over conventional techniques using adjustable radially confining cuffs or sheaths that for example add substantial profile, i.e. diameter, to the operating system—thus the present embodiment is more locally actuated and reduces profile. Such related system is shown for example in FIGS. 14A and 14B to include an electrical source 310 electrically coupled to sacrificial joint 325 of each of multiple tethers 320 placed in series along filter assembly 360 to hold it taught. As shown, an electrode 312 is included as a ground or return electrode, such as using a patch electrode on the patient's back. A circuit is thus made using the patient's body between the sacrificial joint(s) 325 (exposed portion of conductor otherwise shielded or insulated on other regions), the return electrode 312, and the source 310. Typically RF current is used for dissolution of the joint, whereas direct current may be superimposed in some circumstances, such as for diagnostic purposes for example to indicate when detachment is complete. In the embodiment shown in FIG. 14A and accompanying FIG. 15, tether assembly 320 is closed for delivery of the filter assembly 360. As shown in FIG. 14B in a subsequent mode upon applied current, the dissolution of the joints 325 release the circumferential tether assembly and support structure 364 expands to open the filter assembly 360.

The disclosures of the following issued U.S. patents, and in particular without limitation to the extent providing more detailed examples of electrically dissolved medical device implant detachment systems and methods as variously disclosed in one or more of these references, are herein incorporated in their entirety by reference thereto: U.S. Pat. No. 5,851,206 to Guglielmi et al.; U.S. Pat. No. 5,855,578 to Guglielmi et al.; U.S. Pat. No. 5,895,385 to Guglielmi et al.; U.S. Pat. No. 5,919,187 to Guglielmi et al.; U.S. Pat. No. 5,925,037 to Guglielmi et al.; U.S. Pat. No. 5,928,226 to Guglielmi et al.; U.S. Pat. No. 5,944,714 to Guglielmi et al.; U.S. Pat. No. 5,947,962 to Guglielmi et al.; U.S. Pat. No. 5,947,963 to Guglielmi; U.S. Pat. No. 5,976,126 to Guglielmi; U.S. Pat. No. 5,984,929 to Bashiri et al.; U.S. Pat. No. 6,010,498 to Guglielmi; U.S. Pat. No. 6,015,424 to Rosenbluth et al.; U.S. Pat. No. 6,066,133 to Guglielmi et al.; U.S. Pat. No. 6,086,577 to Ken et al.; U.S. Pat. No. 6,156,061 to Wallace et al.; U.S. Pat. No. 6,165,178 to Bashiri et al.; U.S. Pat. No. 6,193,708 to Ken et al.; U.S. Pat. No. 6,375,669 to Rosenbluth et al.; U.S. Pat. No. 6,425,893 to Guglielmi; U.S. Pat. No. 6,425,914 to Wallace et al.; U.S. Pat. No. 6,468,266 to Bashiri et al.; U.S. Pat. No. 6,658,288 to Hayashi; and U.S. Pat. No. 6,716,238 to Elliott. The disclosures of these references are herein incorporated in their entirety by reference thereto.

For further illustration of other features provided among the embodiments of FIGS. 14A-17, the filter assembly 360 is further shown to include a guidewire tracking lumen 372 within delivery member 370, a filter wall 363 with a filter membrane 361 that has engineered porosity for example that may be for example according to the other embodiments of the present invention. Also included is a filtering pouch or cavity 365 formed that is open at proximal end 362 that includes the support ring 364 and that is closed at distal end 366. The system works over a guidewire 340, according for example to the disclosure of WO 2004/039287 to Peacock et al. herein incorporated in its entirety by reference thereto.

It is to be appreciated according to various of the foregoing embodiments that an embolic filter system according to the present invention provides various substantial benefits over previously disclosed systems and methods in the field.

It is to be also appreciated, however, that the present invention may provide such benefit either on its own accord, or in further combination with other features or embodiments of other disclosures or otherwise available or obvious to one of ordinary skill. And, furthermore, such additional combinations constitute further embodiments hereof.

Such additional combinations contemplated hereunder include one or more of the present aspects, modes, embodiments, variations, or features in combination with one or more of those disclosed in co-pending published PCT Patent Application No. WO 2004/039287 to Peacock et al., which is herein incorporated in its entirety by reference thereto.

In general according to these additional aspects, a filter assembly is provided that has a guidewire tracking assembly. This guidewire tracking assembly is adapted to slideably engage a guidewire initially placed across a vascular occlusion (or otherwise to a site where filtering is to be performed). The guidewire tracking assembly in a radially collapsed condition is advanced by a delivery assembly to slide or “shuttle” over the distally seated guidewire and follow the guidewire to the distal filtering location past the vascular occlusion. The filter assembly includes an adjustable lock assembly that is adjustable between an open position, which allows the filter assembly to shuttle over the guidewire, to a locked position, which locks the filter assembly onto the guidewire in situ at the distal location past a vascular occlusion. Once locked onto the guidewire, the filter is adjustable to the radially expanded condition and is detachable from the delivery assembly and thus becomes a part of the guidewire in-situ at the distal location. Thereafter the filter assembly is adapted to be withdrawn in unison with the guidewire and to be groomed into a captured configuration within a capture sheath.

According to further more detailed aspects providing for such combinations, a loop-shaped support member is generally housed within a circumferential passageway formed within a filter member wall. The support member is self-adjustable from a radially collapsed condition to a radially expanded condition that generally correspond with radially collapsed and expanded configurations for the filter member wall. The support member is a memory alloy metal and self-adjusts to the radially expanded condition according to material recovery from a deformed condition of the material corresponding with the radially collapsed condition to a memory condition. The support member is adjusted to the radially collapsed condition within a radial constraint, such as within a delivery lumen of a delivery or guide sheath.

As shown in FIG. 15, the filter module 314 and guidewire 340 may be locked together and coupled prior to use in-vivo, whereas the filter module 314 is adjusted relative to the longitudinal axis L of delivery sheath 350 so as to be positioned within delivery lumen 356. This collapses adjustable filter member 360 from a radially expanded condition to a radially confined condition shown in FIG. 15. FIG. 15 shows certain further detail of one embodiment for filter member 360 for further illustration, and shows a collapsed configuration for a proximal support member 324 and folded filter wall 322. Proximal support member is for example a ring-shaped support member that is constructed of a superelastic alloy material, such as a nickel-titanium material, having a memory shape corresponding the a radially expanded configuration that further corresponds to the expanded condition of the filter member. Filter wall 322 is for example a porous sheet of material, or other filter membrane or structure. Further aspects of these respective components will be explained in further detail by reference to other exemplary embodiments below.

It is to be appreciated therefore that the embodiment illustrated by FIGS. 1A-D provide a beneficial ability to customize the position of a filter assembly along a guidewire, such as at a location along its length relative to other structures such as the distal tip of the guidewire 340. This allows the ability to customize the filtering location in reference to a desired placement of the guidewire 40 in the body. Moreover, the filter may be used with a variety of different guidewires, such as stiffer, more flexible, varied tip shapes, varied diameter sizes, materials, etc. The physician is not required to use a particular guidewire provided with the filter. Thus, particular anatomical or procedural concerns specific to a patient intervention may be met with the ability to customize the filtering device. Still further, this arrangement nevertheless allows the guidewire and filter assembly to be integrated ex-vivo prior to the intervention, providing certain other benefits including for example the potential to achieve lower profiles than certain other “over-the-wire” filtering assemblies and techniques that track over a guidewire in-vivo.

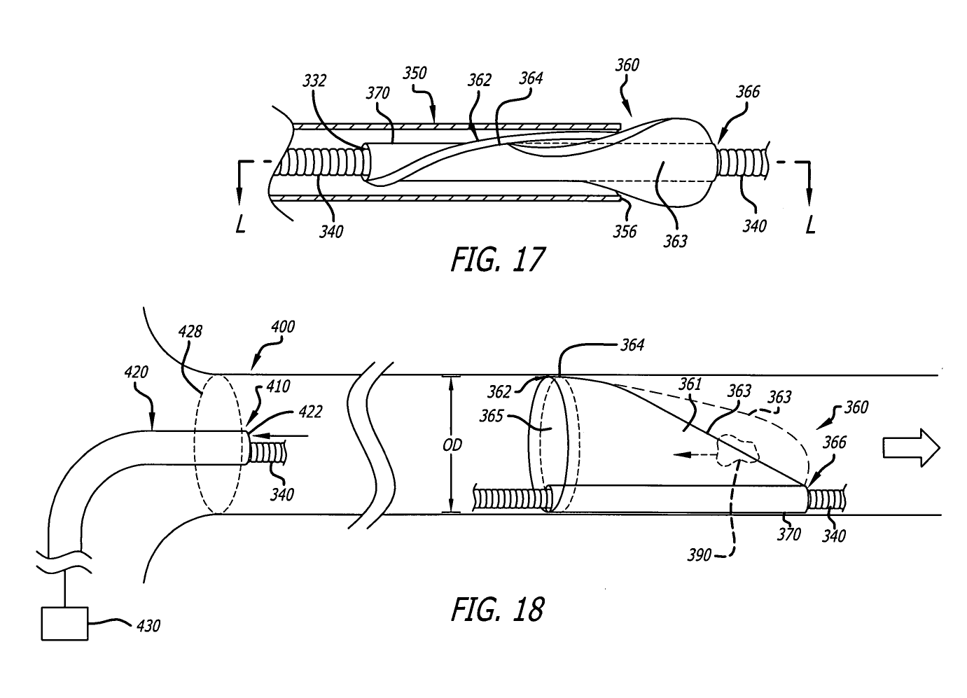

FIGS. 16 and 17 show further detail of a filter module 360 according to one more particular embodiment as follows, and is shown after being locked and detached onto guidewire 340, and before (FIG. 16) and after (FIG. 17) being radially confined within a delivery lumen 356 of a delivery sheath 350.

More specifically, FIG. 16 shows filter member 361 in a radially expanded condition externally of sheath 350. A distally tapering circumferential wall 363 extends between an open proximal end 362, where it is supported by a ring or “loop”-shaped support member 364, and a distal end 366, where it is secured onto tubular support spine 370 that is locked onto wire 340 within inner lumen 372. In the radially expanded configuration shown in FIG. 16, distally extended from delivery sheath 350, the filter member 361 thus provides a pocket 365 that is open along proximal end 362, and closed at distal end 366. Wall 363 is substantially porous to such that normal physiologic blood components flowing into the pocket 365 will pass through wall 363, but whereas debris above a pre-determined dimension, such as from upstream (e.g. proximal relative to the module 60) interventions, will not pass and be captured within pocket 365.

FIG. 17 shows engagement of the module 360 within delivery lumen 356 of delivery sheath 350 subsequent to forming a filtering operation and with certain debris captured within filter member 361. As shown in one particular illustrative mode, such debris may provide increased profile to the collapsed condition of filter module 360, and thus it may be only partially engageable within the radially confining lumen 356 of sheath 350. However, in such circumstance, such may be removed as a system from the body, with the debris successfully filtered, captured, and removed.

FIG. 17 further shows more detail of the relationship between proximal support member 364 and its radially collapsed condition in the radially collapsed configuration for module 360 within delivery lumen 356 of sheath 350. Sheath 350 essentially grooms ring or “loop”-shaped support member 364 into a relatively linear orientation along longitudinal axis L, and radially collapses the otherwise open ring to a radially collapsed condition. This orientation allows for sufficient real estate within delivery lumen 356 to house support member 364 in the collapsed condition. Support member 364 may be provided in a slightly canted orientation in the radially expanded condition outside of sheath 350 in order to accommodate smooth relative advancement of sheath 350 over the ring-shape during the grooming process of radial engagement within lumen 356.

Support member 364 may be coupled to the annular end of the material sheet forming filter member 361 in a variety of modes apparent to one of ordinary skill, though the particular beneficial mode shown herein is described as follows for illustration (and sharing various similar description and relationship with other embodiments elsewhere herein described). The annular end 362 includes a circumferential pouch formed by inverting or everting the end of the material sheet forming filter member 361 on itself and then bonding the inverted or everted edge to the wall, such as by heat bonding, material welding, solvent bonding, adhesive bonding, stitching, etc. the loop-shaped support member 364 may be positioned so as to be captured within the pouch as it is formed, or may be thereafter inserted therein, such as by leaving or forming un-bonded portions, e.g. apertures or ports into the pouch. This all may be accomplished for example by forming the member initially as a flat sheet and providing support member 364 as a partial looped region between two opposite free wire ends. Such arrangement leaves two opposite openings to the inverted or everted pouch along an axis at the edge of the sheet transverse to a long axis of the sheet. One of the top opposite free wire ends is inserted into the pouch and strung therethrough until the partial loop-shaped region is positioned within the pouch. By bringing the free opposite ends together, they may be bonded either together or to the support spine or tubing 370. In this arrangement, such free ends may be in a bent orientation transverse to the plane of the radius of curvature for the intermediate loop located within the pouch. In any case, the opposite longitudinal edges of the sheet are also brought together to form the partial tubular member, and may be either bonded together or bonded to spine 370 to form the filter module 360. In this arrangement, of course the sheet may be either post-processed, or cut along a pre-arranged correlate pattern, that allows for the shaped taper toward the distal end 66 which is rendered in a closed condition and secured to guidewire tracking and support spine 370.

The radially collapsed condition for support member 364 corresponds to a radially collapsed configuration for the overall filter assembly or module 360, which further includes a folded orientation for filter member 361. The radially expanded condition for support member 364 corresponds to a radially expanded configuration for filter assembly module 360, which includes an orientation for filter member 61 that spans across a substantial cross-section of the respective lumen within which it is deployed.

In the particular beneficial embodiments shown, support member 364 is a material having substantial shape member, such as a metal alloy such as nickel-titanium alloy that demonstrates either shape member under thermal changes, or superelastic shape memory, during the change of conditions for the component. For example, the radially collapsed condition corresponds with a deformed condition of the material from a memory condition. The support member 364 is kept in the deformed condition within radially confining forces of tether assembly 320. Upon release therefrom, the force of radial confinement is removed, and thus support member 364 self-adjusts to the radially expanded or extended condition according to material recovery to the memory condition. Such memory condition and related memory shape may correspond with the shape shown for the radially expanded condition, or the memory shape may be something different and the support member 364 is still under some constraint or deformation therefrom even in the radially expanded condition. For example, the vessel wall itself may provide such restraint, and in fact such may allow for a range of lumens to be appropriately treated, as the support member 364 under external wall constraint may have varied radially expanded conditions with shapes on planes with different angles transverse to the longitudinal axis of the lumen in order to span the cross section of different diameters of lumens.

In any event, when appropriate according to a treating physician, after a procedure the distal filter assembly is adjusted back to a radially collapsed condition to capture the emboli filtered from the downstream blood flow. This may be done by again advancing a radially confining sheath over the wire and over the filter, such as by using the first control system a second time, or with a second outer sheath. Or, a pull wire or multiplicity thereof may be used to pull down support member(s) supporting the filter assembly in the expanded configuration. Depending upon the amount of emboli captured, all of the collapsed filter assembly may not be small enough to fit into an outer sheath, which case the entire system may need to be withdrawn over the guidewire and from the body. Otherwise, the collapsed filter may be withdrawn through the outer sheath, or filter and outer sheath together withdrawn within a guiding catheter guide lumen.

As described above, following filtering operation, a grooming sheath 350 may be used to collapse the filter 360 with filtered contents. However, in further embodiments not shown, the tethers may be integrated or coupled with the filter to retract it down for withdrawal. In further embodiments, the tether may include a mechanical coupling that is adjustable between a locked mode that holds the tether taught around the filter frame, and a release mode that releases the filter assembly frame from radial confinement. This may include for example thread tethers that loop around the filter assembly with both free ends held within a delivery catheter, but whereas releasing one end and pulling on the other, the loop is unthreaded. In another mode, the elongate body of the delivery member may include a guidewire; or, the elongated body of the delivery member is a tubular guidewire tracking member in still other embodiments.

A further embodiment of the invention providing substantial further benefit to reduce the need or concerns about management of contents captured within a distal embolic filter is illustrated in FIG. 18 and described as follows.

FIG. 18 shows a similar distal embolic filter assembly 360 to that shown in FIG. 16, except in the open configuration in-vivo within a vessel 400 such as a carotid artery. In this configuration, blood is allowed to flow through the filter 360, whereas debris such as embolism 390 is prevented from flowing through the filter 360. Also provided is a proximal filter assembly 410 that includes an end-hole suction catheter 420 with an aspiration lumen 422 that is coupled to a suction or vacuum source 430. According to this arrangement, Filter assembly 360 is used substantially as previously describe above during a filtering procedure. However, during the procedure, and in any event prior to removal following a procedure, the proximal filter assembly 410 is used to reverse flow in vessel 400 to clear the contents of filter assembly 360, such as for example embolism 390. Otherwise, embolism 390 becomes obstructive to flow through filter assembly 360 potentially causing hemolysis, or otherwise becomes a nidus for further clot formation. Moreover, such content clearance prior to filter removal reduces the profile of the filter to fit through smaller delivery catheters and with lower traumaticity to the vascular anatomy during the removal process. Further shown in FIG. 18 is a balloon 428 (shown in shadow) that may be included for aspiration catheter 410 to assist in achieving the desired suction and flow reversal through vessel 400 sufficient to clear filter 360 of its contents.

As discussed in other portions of the present disclosure, the present embodiments that are herein shown and described in various levels of detail are considered applicable in combination with other embolic filter assemblies otherwise heretofore disclosed in the art to the extent modified appropriately for combination assemblies and mode of operation consistent with this disclosure. In particular, the present embodiments are considered highly beneficial for use in distal embolic filtering, such as in distal filtering of emboli during carotid artery interventions such as stenting, endarterectomies, angioplasty, atherectomy, thrombectomy, etc., or distal filtering distal to saphenous vein graft interventions.

The various embodiments described above are generally intended for use in overall embolic filtering systems intended to be used in cooperation with other devices to filter primarily emboli from blood flowing through vessels downstream from an intervention site. Certain reference is made to specific beneficial applications for the purpose of illustration, but such specified applications are not intended to be limiting. For example, reference to the embolic filters of the invention is often specified for use in distal filtering downstream from interventions as the most frequent type of filtering used in conventional interventions. However, other filters for all uses may be made according to the various embodiments herein described, including for example proximal filters. In addition, it is also contemplated that other regions of the body may be effectively filtered than those specifically described herein, such as other body lumens including for example veins, gastrointestinal lumens, urinary lumen, lymph ducts, hepatic ducts, pancreatic ducts, etc. In addition, whereas many different filters may be used, the coupling of filters to guidewire tracking or locking chassis per the embodiments may be done by any conventional acceptable substitute modes. In addition, various locking mechanisms have been described for purpose of providing a detailed illustration of acceptable modes of making and using the invention. However, other locking modes may be employed without departing from the scope of the invention.

Where “proximal” or “distal” relative arrangements of components, or modes of use, are illustrated, other arrangements are contemplated though they may not be shown. For example, where various of the embodiments are adapted for antegrade use, they may be modified for retrograde delivery and use. In addition, proximal filtering may be accomplished according to the invention, such as by positioning a filter device proximal to an occlusion and using applied retrograde flow to wash emboli proximally into the filter.

Various modifications may be made to the previously disclosed embodiments above without departing from the scope of the present invention which is intended to be read as broad as possible with regard to the intended objectives described herein and without impinging upon what is already known in the art. Many examples of such modifications have been provided as illustrative and are not intended to be limiting, though significant value may be had in relation to certain such specific modifications or embodiments. Where particular structures, devices, systems, and methods are described as highly beneficial for the primary objective herein to provide adjustable embolic filters, other applications are contemplated both in medicine and otherwise in and out of the body. For example, various of the membrane materials of engineered porosity and local bioactivity herein described may be found highly beneficial for use as improved materials for use with other devices and assemblies, either as filters or otherwise. In another example, various specific applications may benefit from the methods herein disclosed of using electroless or other metallic deposition onto polymer substrates, e.g. onto catheter chassis or other operable device components, for the purpose of masking for photoablation of engineered patterns.

The various detailed descriptions of the specific embodiments may be further combined in many differing iterations, and other improvements or modifications may be made that are either equivalent to the structures and methods described or are obvious to one of ordinary skill in the art, without departing from the scope of the invention. The illustrative examples therefore are not intended to be limiting to the scope of the claims below, or with respect to the Summary of the Invention, unless such limitation is specifically indicated.

1. The system of claim 2, further comprising:
a delivery member with an elongate body;
wherein the wall is mounted on a super-elastic, nickel-titanium frame;
wherein the frame has a memory in a radially expanded condition, and is self-expandable from a radially collapsed condition to a radially expanded condition;
wherein the frame is held in radial confinement in the radially collapsed condition by at least one releasable circumferential tether that holds the frame substantially tight around the elongated body of the delivery member; and
wherein the tether is releasable at the distal location to thereby remove the radial confinement on the frame and allow the frame to self-expand to the radially expanded condition.

2. An embolic filter system, comprising:
a distal embolic filter assembly with a wall that is adapted to be delivered to a and span across a distal location within a vessel in a patient and that is substantially porous so as to filter emboli from antegrade blood flowing to and through the wall at the distal location;
a plurality of discrete apertures through the wall and providing the substantial porosity; and
wherein each of the plurality of apertures comprises a geometry with a length and a width, and the length being substantially longer than the width.

3-6. (canceled)

7. The system of claim 2, further comprising:
a delivery member with an elongate body;
wherein the distal embolic filter assembly is coupled to the delivery member for delivery to the distal location.

8-11. (canceled)

12. The system of claim 7, wherein:
the delivery member comprises a guidewire tracking member and is adapted to track over a guidewire to the distal location.

13. The system of claim 7, wherein:
the delivery member comprises an adjustable lock that is adjustable between an open condition, wherein the delivery member is adapted to track over a guidewire, to a locked condition, wherein the delivery member is adapted to lock onto the guidewire such that the guidewire and filter assembly are adapted to be removed from the patient together through a delivery sheath.

14. The system of claim 12, wherein:
the delivery member comprises a distal delivery assembly and a detachable proximal delivery assembly coupled to the distal delivery assembly at a detachable joint;
the distal embolic filter assembly is coupled to the distal delivery assembly; and
the distal delivery assembly is adapted to be positioned entirely within the patient, and the proximal delivery assembly is adapted to extend exernally of the patient, and the proximal delivery assembly is adapted to be released from the distal delivery assembly when the distal embolic filter assembly is positioned at the distal location.

15. The system of claim 14, wherein the detachable joint comprises an electrolytically detachable joint.

16. The system of claim 2, wherein:
the length is at least about twice the width.

17. The system of claim 2, wherein:
the width is equal to or less than about 120 microns.

18. The system of claim 2, wherein:
the length is at least about twice the width; and
the width is equal to or less than about 120 microns.

19-20. (canceled)

21. The system of claim 2, wherein:
the length is equal to or greater than about 120 microns.

22-24. (canceled)

25. The system of claim 2, wherein:
the plurality of apertures comprises at least one elongate groove through the wall and bridged by filaments; and
the geometry is defined by distance between the lateral edges of the groove and the spacing between the filaments.

26. The system of claim 25, comprising a plurality of said grooves, each extending longitudinally along a substantial portion of the length of the wall.

27. The system of claim 25, comprising a plurality of said grooves, each extending circumferentially around a long axis of the filter wall.

28. The system of claim 25, wherein said groove comprises a helical shape along a length and circumference of the filter wall.

29. (canceled)

30. The system of claim 2, wherein:
the wall comprises a composite structure with a polymer membrane in combination with a network of structural support struts;
the network of structural support struts is coupled to the membrane;
wherein the plurality of apertures communicate through the membrane; and
wherein at least one of the structural support struts spans across each of the apertures.

31-33. (canceled)

34. The system of claim 30, wherein:
the network of structural support struts comprises a plurality of metallic filaments.

35-37. (canceled)

38. The system of claim 2, further comprising:
a proximal filter assembly with an aspiration catheter and that is adapted to be fluidically coupled to the distal embolic filter assembly at the distal location and to reverse flow at the distal location so as to aspirate contents captured on an upstream side of the embolic filter and to remove said contents from the patient.

39. The system of claim 38, wherein the aspiration catheter further comprises an inflatable balloon.

40-41. (canceled)

42. The system of claim 2, wherein:
the wall comprises a surface that is exposed to the blood at the distal location; and
a bioactive agent is coupled to the surface in a manner expressing substantial bioactivity with respect to the blood in contact with the surface.

43-47. (canceled)

48. The system of claim 42, wherein:
the surface comprises a drug eluting matrix carrier that is different than the bioactive agent and that holds and elutes the bioactive agent.

49-62. (canceled)

63. The method of claim 64, further comprising:
providing a delivery member with an elongate body;
mounting a substantially porous wall on a super-elastic, nickel-titanium frame that is secured to the elongated body;
providing the frame to have a material shape memory in a radially expanded condition, such that the frame is self-expandable from a radially collapsed condition to a radially expanded condition;
holding the frame in radial confinement in the radially collapsed condition by at least one releasable circumferential tether that holds the frame substantially tight around the elongated body of the delivery member; and
releasing the tether at the distal location to thereby remove the frame from radial confinement and allow the frame to self-expand to the radially expanded condition;
wherein the distal embolic filter wall comprises the substantially porous wall.

64. A method for manufacturing an embolic filter system, comprising:
forming a plurality of discrete apertures through a distal embolic filter wall such that a length of each aperture is substantially longer than the width of the respective aperture.

65. The method of claim 64, further comprising:
coupling a network of structural support struts to a membrane constructed from a polymer matrix to thereby form a composite structure;
forming a plurality of apertures that communicate through the membrane and such that at least one of the structural support struts spans across each of the apertures; and
using the composite structure with apertures formed therethrough as the distal embolic filter wall for a distal embolic filter assembly.

66. The method of claim 64, further comprising:
providing the distal embolic filter assembly;
providing a proximal embolic filter assembly;
wherein the distal and Proximal embolic filter assemblies are useful in combination by:
conducting a distal embolic filter procedure at a distal location within a blood vessel in a patient using the distal embolic filter assembly such that antegrade flow perfuses through a substantially porous wall of the distal embolic filter assembly but further such that material is captured at an upstream side of the filter wall; and
conducting a proximal embolic filter procedure on the patient by using the proximal embolic filter assembly to reverse flow at the distal location such that the the material captured at the upstream side of the filter wall is flushed proximally into an aspiration lumen and sheath at a proximal location associated with the vessel.

67. The method of claim 42, further comprising:
coupling a bioactive agent to a surface of the distal embolic filter wall, wherein the bioactive agent is a different material than a polymeric membrane of the distal embolic filter wall; and
expressing substantial bioactivity with respect to blood in contact with the surface using the bioactive agent.

68. (canceled)
